(19) United States
(12) Patent Application Publication
(10) Pub. No.: US 2007/0184537 A1
(43) Pub. Date: Aug. 9, 2007

(54) Method for the linkage of bifunctional chelating agents and (radioactive) transition metal complexes to proteins and peptides

(75) Inventors: Roger Schibli, Baden (CH); Albert Stichelberger, Morges (CH); Robert Waibel, Raeterschen (CH); August Schubiger, Lengnau (CH)

Correspondence Address: SIEMENS SCHWEIZ AG;I-47, INTELLECTUAL PROPERTY, ALBISRIEDERSTRASSE 245, ZURICH, CH-8047, CH

(73) Assignee: Paul Scherrer Institut, Villigen (CH)

(21) Appl. No.: 10/557,893

(22) PCT Filed: Jun. 2, 2004
(86) PCT No.: PCT/EP04/05964

§ 371 Date: Mar. 6, 2007

(30) Foreign Application Priority Data
Jun. 3, 2003 (EP) 03076728.9

(51) Int. Cl. C07D 213/06

(52) U.S. Cl. 435/131; 540/465; 546/329

(57) ABSTRACT

The present invention relates to a method for radioactive labeling of a protein or peptide, by providing a protein or peptide having at least one glutamine or lysine residue; adding a metal chelating agent having at least one lysine or glutamine residue, respectively, which metal chelating agent is optionally complexed with a radioactive or paramagnetic metal; reacting the protein or peptide and metal chelating agent in the presence of a transglutaminase to obtain a protein or peptide with a metal chelating group covalently bound thereto, and optionally complexing the metal chelating group with a radioactive or paramagnetic metal. The invention also relates to proteins and peptides thus labeled and to proteins and peptides that have been coupled to a metal chelating agent but not yet labeled.

### FIELD OF THE INVENTION

The present invention relates to a method for the functionalization of proteins, peptides and other biologically active molecules with metal chelating agents, in particular bifunctional transition metal chelating agents (BFCA), and to (radioactive) metal complexes based thereon. The invention also relates to bifunctional transition metal chelating agents for use in the method.

### BACKGROUND OF THE INVENTION

Radioactively labeled monoclonal antibodies (mAb), antibody fragments (scFv) and peptides are very important molecules for diagnosis and therapy of cancer. But also other proteins can be radioactively labeled and applied in diagnosis and therapy or otherwise. The site-specific radiolabeling of large proteins such as monoclonal antibodies and antibody fragments with sufficient high specific activity for therapy remains, however, a problem.

Direct labeling of proteins with radiometal isotopes e.g. Tc-99m, Re-186/188, is often performed using reducing agents in the reaction solution. This may lead to the proteins at least partially losing their biological activity, to misfolding and to metal incorporation at the protein's active site. Chemical modification of proteins with a bifunctional chelating agent for stable in vivo coordination of radionuclides (post-labeling approach) or the modification with pre-formed radioactive transition metal complexes (pre-labeling approach) is difficult and may have the same impact on the biological activity for the same reasons mentioned above. Furthermore, most of these techniques are not site-specific and some need elevated temperatures to achieve sufficient high specific activities. Thus, reaction of heat sensitive peptides and proteins often leads to denaturation and loss of their biological activity.

### SUMMARY OF THE INVENTION

It is a first object of the-present invention to provide a site-specific labeling and functionalization method, that is particularly intended for heat and chemically sensitive proteins, and that does not have the above stated drawbacks. Such a technique would be of high significance for a more widespread application of such radiolabeled proteins and peptides in the diagnosis and therapy of cancer and other diseases or in other applications. The technique is intended to be versatile in respect of metal chelating systems and radionuclides.

According to the invention, the functionalization of the biologically active molecules is site-specific and occurs via, respectively between lysine and glutamine residues of the corresponding reaction partners by transglutaminase. Enzyme mediated radiolabeling of proteins, has so far not yet been reported.

### DETAILED DESCRIPTION OF THE INVENTION

The present invention thus relates to a method for radioactive labeling of a protein or peptide comprising the steps of:

a) providing a protein or peptide having at least one glutamine or lysine residue;

b) adding a metal chelating agent having at least one lysine or glutamine residue, respectively;

c) reacting the protein or peptide and metal chelating agent in the presence of a transglutaminase to obtain a protein or peptide with a metal chelating group covalently bound thereto; and

d) complexing the metal chelating group with a radioactive or paramagnetic metal.

Alternatively, the invention relates to a method for radioactive labeling of a protein or peptide comprising the steps of:

a) providing a protein or peptide having at least one glutamine or lysine residue;

b) adding a metal chelating agent having at least one lysine or glutamine residue, respectively, which metal chelating agent is complexed with a radioactive or paramagnetic metal;

c) reacting the protein or peptide and metal complexed metal chelating agent in the presence of a transglutaminase to obtain a protein or peptide with a radioactively labeled metal chelating group covalently bound thereto.

The first method is a post-labeling method, whereas the second method is based on pre-labeling of the chelating agent. The principle of both methods is however the same, namely coupling of a chelating agent to a peptide or protein via the reaction of a lysine and a. glutamine by means of a transglutaminase.

The enzymatic activity of the transglutaminase family catalyzes an acyl transfer reaction between the γ-carboxamide groups of peptide-bound glutamine residues and various primary amines or ε-amino groups of lysine residues, thus forming isopeptidic bonds which are stable and resistant to chemical, enzymatic, and physical degradation. The function of TGases can be described as incorporation of alkylamine derivatives into specific glutamine residues or vice versa. This specificity has been recognized before and has already been applied successfully for different purposes. For instance it is used in nutritional chemistry for improvement of nutritional value and functional properties of food proteins. However, it was not previously used for labeling purposes.

The inventors now present a very convenient method for the site-specific functionalization and radiolabeling of proteins and peptides under near physiological conditions. The labeling procedure is carried out in a buffered system using mild conditions. Additionally, no aggressive chemicals are used, avoiding misfolding or denaturation of the protein.

The method allows the application of any type of transglutaminase (TGase) for this purpose. Several types of transglutaminases have been reported in various living organisms including microbials. Examples are TGase from guinea pig liver (GTGase), fish liver (FTGase) and microorganisms (MTGase) and any recombinant Tgase (rTGase). Other TGases than the ones listed here can also be used according to the invention.

The target biomolecule has to provide at least one lysine or glutamine residue. The metal chelating agent and the corresponding transition metal complex have to provide at least a glutamyl or a 5-amino pentyl residue, respectively. These lysine and glutamine residues should not be present at or close to the active site(s) of the protein or peptide or else its biological activity could be hampered. The method enables a radioactive pre-labeling as well as a post-labeling approach of proteins and other (bio)molecules (FIG. 1).

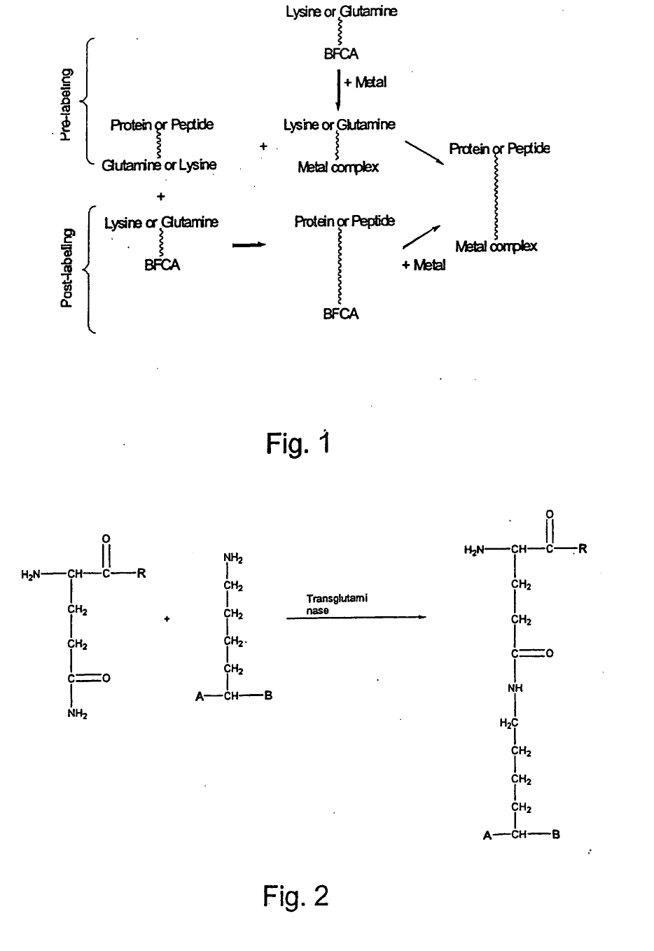

In one embodiment the method allows selective formation of covalently linked conjugates via an ε-amino group of lysine and the γ-carboxamide groups of a glutamine pendent the metal chelating agent or a transition metal complex thereof (FIG. 2).

In this embodiment the lysine is part of the protein or peptide to be labeled and the glutamine is part of the metal chelating agent.

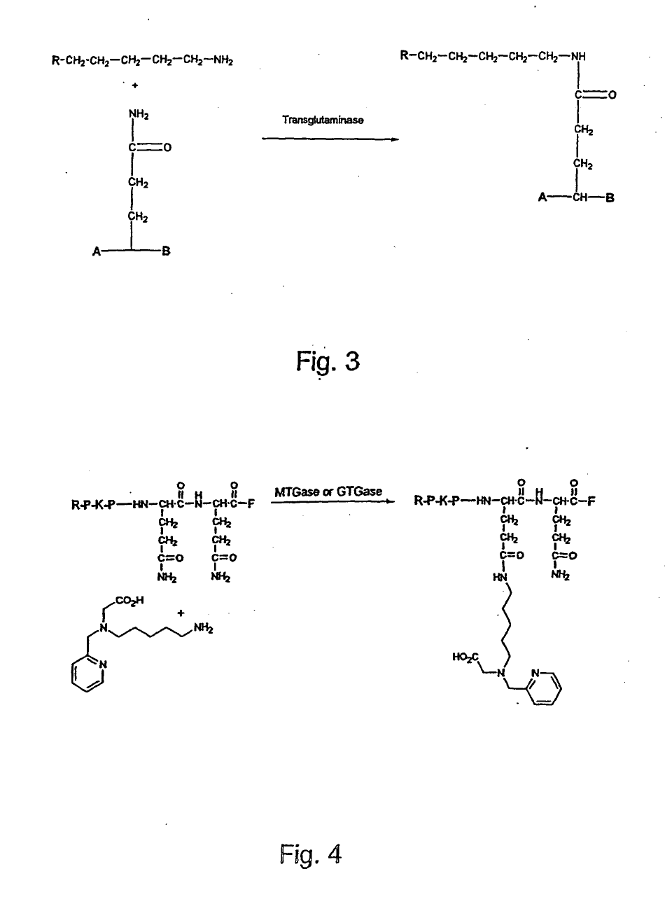

In a second embodiment, the method allows the selective formation of covalently linked conjugates between the γ-carboxamide groups of glutamine and a free pendent primary amino group of a metal chelating agent or a transition metal complex thereof (FIG. 3). In this embodiment the glutamine is part of the protein or peptide and the lysine is part of the metal chelating agent.

The primary amino group is preferably separated by at least five (CH₂)-groups or a spacer of equal length from the metal chelating moiety.

The metal chelating agent has for example the following structure:
R—CH₂—CH₂ —CH₂—CH₂ —CH₂—NH₂
wherein R represents a latent reactive group capable of coordinating to a metal center or a metal complex.

Alternatively, the metal chelating agent has the following structure: 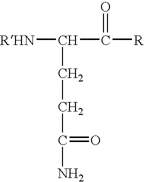
wherein R represents a latent reactive group capable of coordinating to a metal center or a metal complex and wherein R′ represents a H atom or an N-carbobenzoxy group.

In the pre-labeling method, the metal chelating agent has one of the following structures:

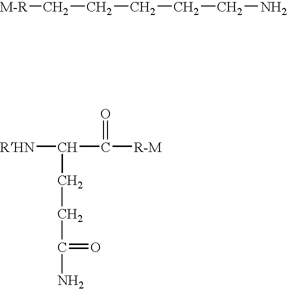

Advantageously, the metal chelating agent is a transition metal chelating agent. The radioactive metal may be any radioactive transition metal isotope and is for example selected from ^{99m}Tc, ¹⁸⁶Re, ¹⁸⁸Re, 64Cu, ⁶⁷Cu, ⁶⁸Ga, ⁶⁸Ge, ⁹⁰Y, ¹⁰⁵Rh, ¹¹¹Ag, ¹¹¹In, ¹⁴⁹Pm, ¹⁵³Sm, ¹⁶⁶Ho, ¹⁶⁹Gd, ¹⁷⁷Lu. The paramagnetic metal may be any paramagnetic transition metal. Examples are Gd(+III), Mn(+II), Mn(+III); Fe(+III) The invention further relates to a radioactively labeled protein or peptide obtainable by means of the claimed method.

According to a still further aspect thereof the invention relates to a bifunctional transition metal chelating agent comprising a metal chelating moiety and a lysine or glutamine side chain. The lysine or glutamine may be incorporated in a larger molecule, such as a protein or peptide.

The metal chelating moiety can be any suitable metal chelating moiety and is for example selected from the group consisting of PAMA (2-picolylamine mono acetic acid), a histidyl group, cysteines, isonitriles, IDA (imino diacetate), DOTA (1,4,7,10-tetraazacyclododecane-1,4,7,10-tetraacetic acid), DTPA (diethylenetriaminepentaacetate), CPTA (4-(1,4,8,11-tetraazacyclotetradec-1-yl)-methyl benzoic acid), HYNIC (6-hydrazinonicotinic).

Particularly useful bifunctional transition metal chelating agents have one of the following formulas: 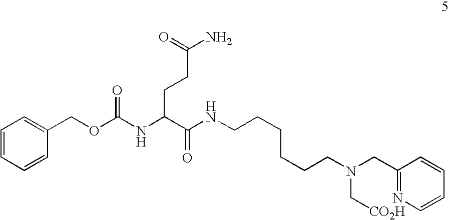
The radioactively labeled bifunctional metal chelating agents of the general formula: 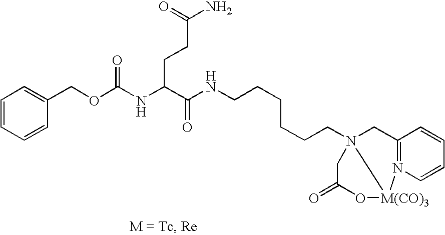
are also part of this invention. Besides Tc and Re, the metal may be any other radioactive or paramagnetic transition metal, for example one of the list mentioned above. They do not need to be in the form of a carbonyl as in this formula.

The invention also relates to compounds of the invention that are labeled with a radioactive or paramagnetic label.

The invention will be illustrated in the Examples that follow. The Examples are for illustration purposes only and are not intended to limit the invention in any way. In the Examples reference is made to the following figures:

FIG. 1: Pre- and post-labeling strategies.

FIG. 2: Selective formation of covalently linked conjugates via a ε-amino group of lysine and the γ-carboxamide groups of a glutamine pendent bifunctional chelating agent (BFCA) or a transition metal complex thereof. R represents a latent reactive group capable of coordinating to a transition metal center or a radioactive or non-radioactive transition metal complex. Either one or both of A and B may be present and each is an organic residue.

FIG. 3: Selective formation of covalently linked conjugates between the γ-carboxamide groups of glutamine and a free pendent primary amino group of a BFCA or a transition metal complex thereof. R represents a latent reactive group capable of coordinating to a transition metal center or a radioactive or non-radioactive transition metal complex. Either one or both of A and B may be present and each is an organic residue.

FIG. 4: Coupling of a BFCA to the peptide Substance P(1-7).

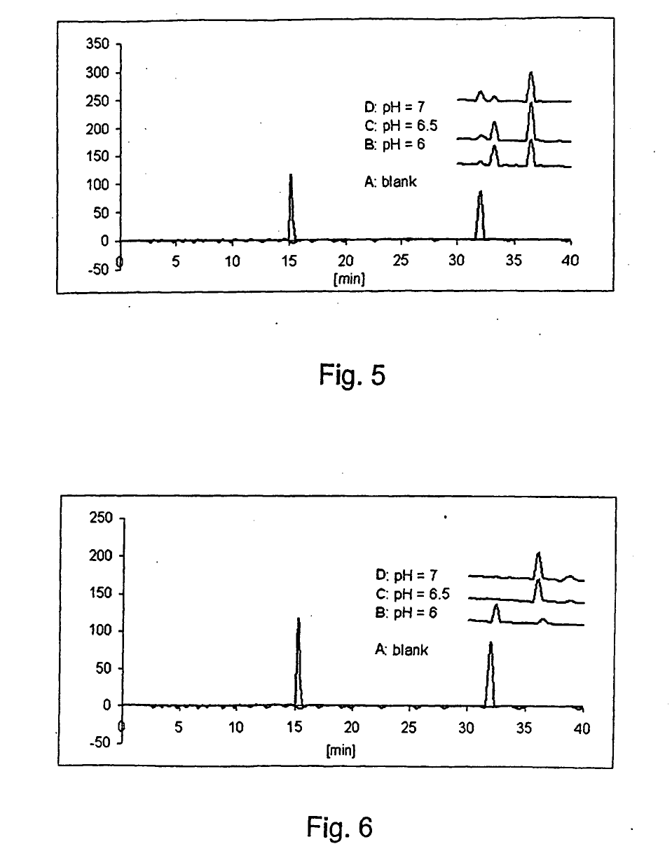

FIG. 5: HPLC UV-chromatograms of GTGase mediated reactions.

FIG. 6: HPLC UV-chromatograms of MTGase mediated reactions.

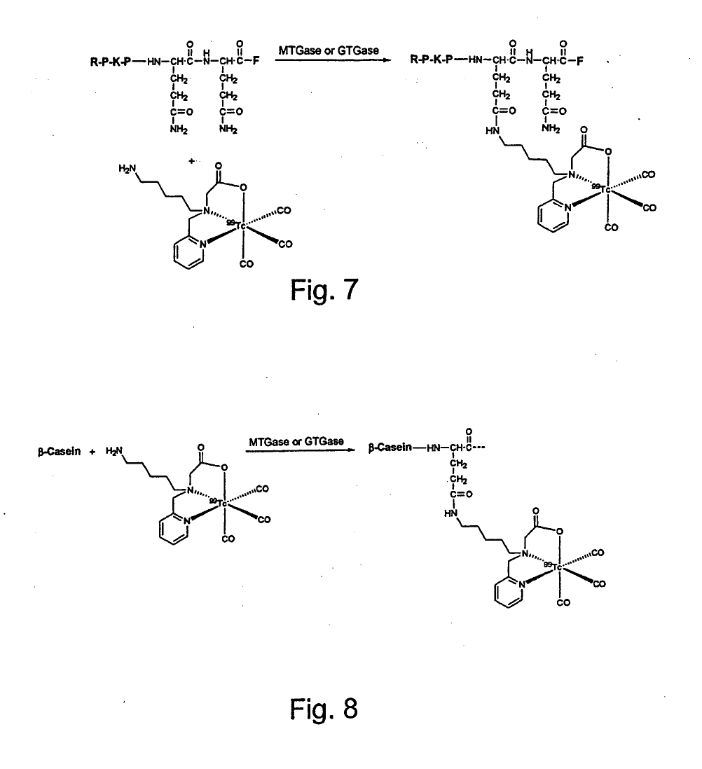

FIG. 7: Coupling of the radioactive transition metal complex [⁹⁹Tc(CO)₃(5-amino-pentyl)-pyridine-2-yl-methyl-amino ]-acetate] to Substance P(1-7) (RPKPQQF).

FIG. 8: Coupling of the radioactive transition metal complex [⁹⁹Tc(CO)₃(5-amino-pentyl)-pyridine-2-yl-methyl-amino ]-acetate] to β-casein.

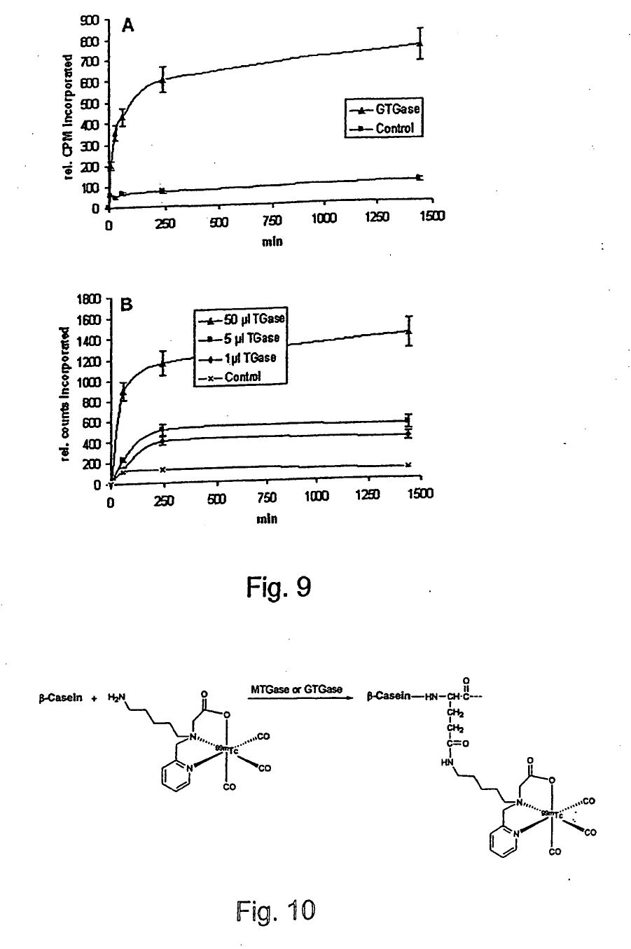

FIG. 9: Incorporation of the ⁹⁹Tc-complex in β-casein.

FIG. 10: Conjugation of the no-carrier added [⁹⁹Tc (CO), (5-amino-pentyl)-pyridine-2-yl-methyl-amino]-acetate] to β-casein.

FIG. 11: Time dependent SDS-Page analysis Conjugation of the no-carrier added [^{99m}Tc(CO)₃(5-amino-pentyl)-pyridine-2-yl-methyl-amino]-acetate] to β-casein by GTGase (upper row) or MTGase (lower row) after 1-5 hours incubation. C=control.

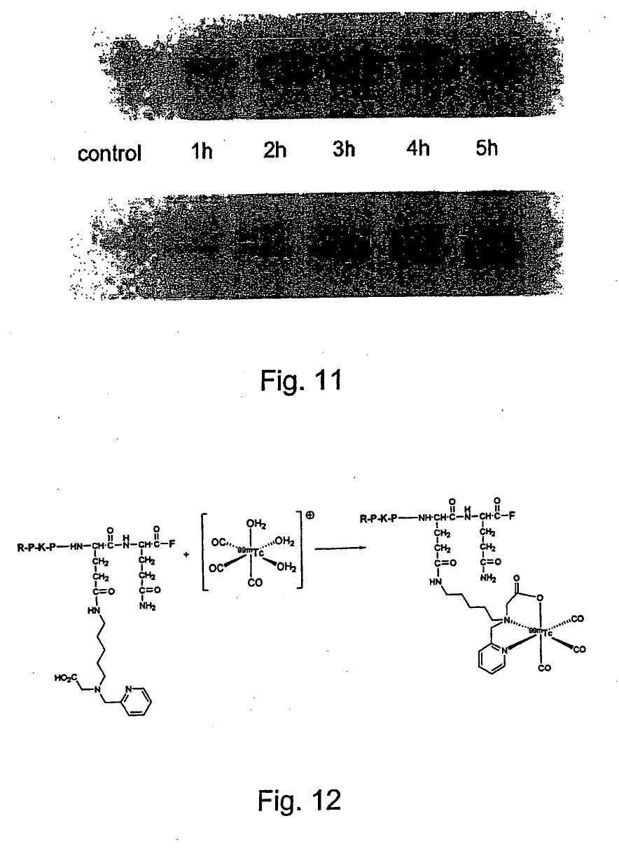

FIG. 12: Post-labeling of substance P(1-7) that was first modified with a BFCA.

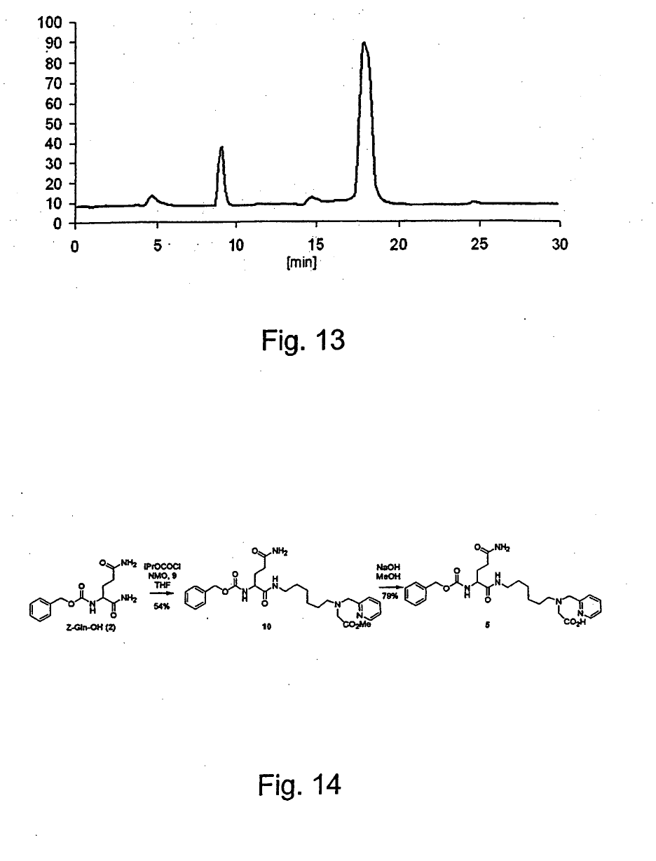

FIG. 13: Radioactive trace of the reaction solution of Example 5.

FIG. 14: Synthesis of compound 5 using isopropyl chloroformiate coupling conditions.

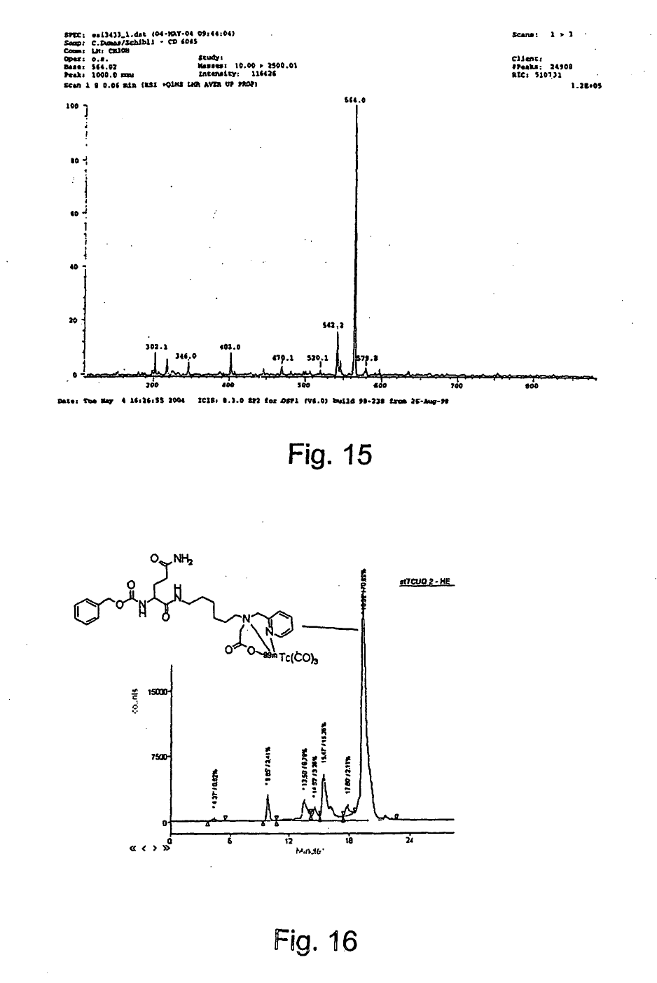

FIG. 15: Mass spectrum of compound 5.

FIG. 16: Labeling of compound 5 at 75° C. after 30 min, 10⁻³M.

### EXAMPLES

### Example 1

### Coupling the BFCA [(5-amino-pentyl)-pyridine-2-yl-methyl-amino ]-acetic acid (APPA) to the peptide Substance P(1-7) (amino acid sequence: RPKPQQF)

The enzymatic activity of GTGase and MTGase was used for coupling the BFCA [(5-amino-pentyl)-pyridine-2-yl-methyl-amino ]-acetic acid (also called herein APPA) to the peptide Substance P(1-7) (amino acid sequence: RPKPQQF) (FIG. 4). Substance P(1-7) was incubated with APPA at pH 6.0, 6.5, and 7 at 37° C. Ca²⁺dependent guinea pig liver transglutaminase (GTGase) or Ca²⁺, independent microbial transglutaminase (MTGase) were used. When MTGase was used, no CaCl₂ was present in the reaction mixture.

The reactions were monitored by means of RP-HPLC. After 4 h the reactions were stopped and the product purified via HPLC. FIG. 5 presents a HPLC UV-chromatograms (λ=254 nm) of the GTGase mediated reactions. The UV trace shows the ligand 1 and Substance P(1-7) with retention time (Rt) of 15.2 min and 32.0 min respectively (FIG. 5A).

In reactions performed at pH=6, 6.5 and 7, two new peaks appeared with Rt of 33.2 min and 36.4 min after 4 hours at 37° C., whereas the peak of Substance P(1-7) at 32.0 min essentially disappeared.

Reactions performed at pH 7 (FIG. 5D), showed the formation of one main product, whereas at acidic pH (pH 6.5 and pH 6) the amount of by-products was significantly higher (FIG. 5B/C).

The HPLC UV-chromatogram of the MTGase mediated reaction showed essentially the formation of solely the desired product APPA-Substance P(1-7) at 36.4 min (FIG. 6). However, this time the pH of the reaction solution had a much more pronounced influence on the overall reaction yield, than in case of GTGase. At a pH of 6 only 15±5% of the product was formed after 4 h as determined via HPLC. However, t pH's 6.5 and 7 the peptides was completely converted into the desired conjugate.

### Example 2

### Coupling the radioactive transition metal complex [⁹⁹Tc(Co)₃(5-amino-pentyl)-pyridine-2-yl-methyl-amino]-acetate] to Substance P(1-7) (RPKPQQF)

The enzymatic activity of GTase and MTGase was used for coupling the radioactive transition metal complex [⁹⁹Tc(CO)₃(5-amino-pentyl)-pyridine-2-yl-methyl-amino]-acetate] to Substance P(1-7) (RPKPQQF) (FIG. 7). Substance P(1-7) was incubated with [⁹⁹Tc(CO)₃(5-amino-pentyl)-pyridine-2-yl-methyl-aminol-acetate at pH 6.0, 6.5, and 7 at 37° C. Ca²⁺dependent guinea pig liver transglutaminase (GTGase) or Ca²⁺independent microbial transglutaminase of MTGase were used. When MTGase was used, no CaCl₂ was present in the reaction mixture.

The reactions were monitored by means of RP-HPLC. After 4 h the reactions were stopped and the product purified via HPLC.

The radioactive samples were collected and analyzed by scintillation counting. Compared to the control, where no MTGase was present, the incorporation of the ⁹⁹Tc-complex was 100 times higher. No significant differences between MTGase and GTGase could be observed.

### Example 3

### Coupling of the radioactive transition metal complex ⁹⁹Tc(CO)₃(5-amino-pentyl)-pyridine-2-yl-methyl-amino)-acetate ] to β-casein

β-casein was incubated with ⁹⁹Tc(CO)₃(5-amino-pentyl)-pyridine-2-yl-methyl-amino]-acetate] (FIG. 8) at pH 7, containing β-mercapto ethanol, EDTA, and CaCl₂ (only for GTGase) and 1-50 mU GTGase or 50 mU MTGase. The reactions were stopped by addition of TFA.

The samples were applied on a fast desalting column for fractionized collection. Aliquots of the protein containing fractions were analyzed by scintillation counting. Compared to the control, where no MTGase was present, incorporation of the ⁹⁹Tc-complex was 10 times higher with highest amount of enzyme after 24 h post incubation (FIG. 9A). For MTGase a similar labeling profile was observed and the maximum incorporation was found to be 7 times higher than in the control experiments 24 h post incorporation (FIG. 9B).

### Example 4

### Conjugation of the no-carrier added ⁹⁹Tc(CO)₃(5-amino-pentyl)-pyridine-2-yl-methyl-amino]-acetate] to β-casein by GTGase or MTGase

β-casein was incubated with [⁹⁹Tc(CO)₃(5-amino-pentyl)-pyridine-2-yl-methyl-amino]-acetate] (FIG. 10) at pH 7, with β-mercapto ethanol, EDTA, CaCl₂, and GTGase or MTGase. Aliquots were taken in intervals of 1-h during the reaction time of 5 h for analysis on SDS-PAGE. SDS-Page analysis revealed that the complex [⁹⁹Tc(CO)₃(5-amino-pentyl)-pyridine-2-yl-methyl-aminol-acetate] is stably attached to β-casein. Time course shows that with progressing time, incorporation of radioactivity into β-casein increased. In the control assay no GTGase was present (FIG. 11).

### Example 5

### Radioactive post-labeling of biomolecule, which is enzymatically modified with BFCA

This example represents the possibility of a radioactive post-labeling of a biomolecule, which is enzymatically modified with BFCA. Purified substance P(1-7)-APPA was incubated with the radioactive precursor [⁹Tc(CO)₃(OH₂)₃]⁺ in physiological saline at 75° C. for 30 min (FIG. 12). FIG. 13 shows the radioactive trace of the reaction solution with the product peak at 36.2 min.

### Example 6

### Preparation of bifunctional metal chelating agent for use in the invention The

glutamine derivative 5 was synthesized by treatment of the protected amino acid 2 with isopropyl chloroformiate in THF and in the presence of 4-methyl-morpholine (NMO) as base (FIG. 14). The coupling reaction occurs in a good yield and the ester was saponified with sodium hydroxid to afford the glutamine ligand 5. The labeling with ^{99m}Tc was carried out and the labeled product was purified directly by mean of preparative HPLC. The mass spectroscopy confirms the formation of compound 5 (FIG. 15).

Labeling of glutamine derivative 5 with ^{99m}Tc (FIG. 16) was furthermore performed in a PBS solution at different temperatures. The formation of the product could be detected at 37° C. after 75 min but the reaction is not complete (data not shown). On the other hand when the reaction is carried out at 75° C., the disappearance of the [^{99m}Tc(CO)₃(H₂O)₃]⁺ complex is total after only 30 minutes (FIG. 16). The glutamine complex could be observed at 19.4 min in a good purity.

1. A method for radioactive labeling a protein or peptide, the method comprising the steps of:
a) providing a protein or peptide having at least one glutamine or lysine residue;
b) adding a metal chelating agent having at least one lysine or glutamine residue, respectively;
c) reacting the protein or peptide and metal chelating agent in the presence of a transglutaminase to obtain a protein or peptide with a metal chelating group covalently bound thereto; and
d) complexing the metal chelating group with a radioactive or paramagnetic metal.

2. A method for radioactive labeling of a protein or peptide, the method comprising the steps of:
a) providing a protein or peptide having at least one glutamine or lysine residue;
b) adding a metal chelating agent having at least one lysine or glutamine residue, respectively, which metal chelating agent is complexed with a radioactive or paramagnetic metal; and
c) reacting the protein or peptide and metal complexed metal chelating agent in the presence of a transglutaminase to obtain a protein or peptide with a radioactively labeled metal chelating group covalently bound thereto.

3. The method according to claim 1, wherein the glutamine is part of the protein or peptide and the lysine is part of the metal chelating agent.

4. The method according to claim 1, wherein the lysine is part of the protein or peptide and the glutamine is part of the metal chelating agent.

5. The method according to claim 2, wherein the glutamine is part of the protein or peptide and the lysine is part of the metal chelating agent.

6. The method according to claim 2, wherein the lysine is part of the protein or peptide and the glutamine is part of the metal chelating agent.

7. The method according to claim 1, wherein the metal chelating agent comprises the following structure:
R—CH₂—CH₂ —H₂ —CH₂ —CH₂ —NH₂
wherein R represents a latent reactive group capable of coordinating to a metal center or a metal complex.

8. The method according to claim 1, wherein the metal chelating agent comprises the following structure: 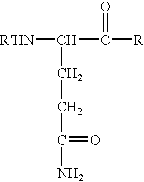
wherein R represents a latent reactive group capable of coordinating to a metal center or a metal complex and wherein R′ represents a H atom or an N-carbobenzoxy group.

9. The method according to claim 2, wherein the metal chelating agent comprises one of the following structures: 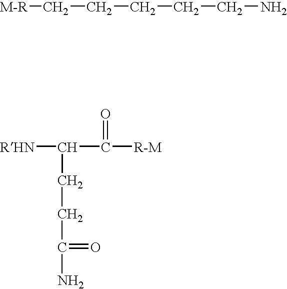
wherein R represents a latent reactive group capable of coordinating to a metal center or a metal complex and wherein R′ represents a H atom or an N-carbobenzoxy group.

10. The method according to claim 1, wherein the metal chelating agent is a transition metal chelating agent.

11. The method according to claim 1, wherein the radioactive metal is selected from a group comprising ^{99m}Tc, ¹⁸⁶Re, ¹⁸⁸Re, ⁶⁴Cu, ⁶⁷Cu, ⁶⁸Ge, ⁹⁰Y, ¹⁰⁵Rh, ¹¹¹Ag, ¹¹¹In, ¹⁴⁹Pm, ¹⁵³Sm, ¹⁶⁶Ho, ¹⁶⁹Gd, and ¹⁷⁷Lu.

12. The method according to claim 1, wherein the paramagnetic metal is selected from a group comprising Gd(+III), Mn (+II), Mn(+III); and Fe (+III).

13. The method a according to claim 2, wherein the metal chelating agent is a transition metal chelating agent.

14. The method according to claim 2, wherein the radioactive metal is selected from a group comprising ^{99m}Tc, ¹⁸⁶Re, ¹⁸⁸Re, ⁶⁴Cu, ⁶⁷Cu, ⁶⁸Ge, ⁹⁰Y, ¹⁰⁵Rh, ¹¹¹Ag, ¹¹¹In, ¹⁴⁹Pm, ¹⁵³Sm, ¹⁶⁶Ho, ¹⁶⁹Gd, and ¹⁷⁷Lu.

15. The method according to claim 2, wherein the paramagnetic metal is selected from a group comprising Gd(+III), Mn(+II), Mn(+III); and Fe (+III).

16. (canceled)

17. (canceled)

18. A bifunctional transition metal chelating agent comprising a metal chelating moiety and a lysine or glutamine side chain.

19. The bifunctional transition metal chelating agent accord to claim 18, wherein the metal chelating moiety is selected from a group comprising PAMA (2-picolylamine mono acetic acid), a histidyl group, cysteines, isonitriles, IDA (imino diacetate), DOTA (1,4,7,10-tetraazacyclododecane-1,4,7,10-tetraacetic acid), DTPA (diethylenetriaminepentaacetate), CPTA (4-(1,4,8,11-tetraazacyclotetradec-1-yl)-methyl benzoic acid), and HYNIC (6-hydrazinonicotinic).

20. The bifunctional transition metal chelating agent according to claim 18, wherein the metal chelating agent comprises the formula:

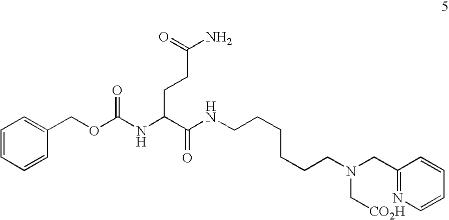

21. A radioactivey labeled bifunctional metal chelating agent comprising the general formula:

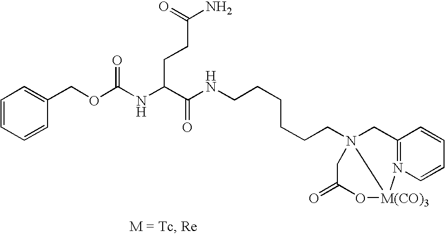

22. The radioactively labeled bifunctional metal chelating agent according to claim 24, wherein M(CO)₃ in the formula is replaced by another radioactive or paramagnetic transition metal.
